(19) United States
(12) Patent Application Publication
(10) Pub. No.: US 2019/0307396 A1
(43) Pub. Date: Oct. 10, 2019

(54) DEVICE AND METHOD FOR DETECTION OF CANNABIS AND OTHER CONTROLLED SUBSTANCES USING FAIMS

(71) Applicants: CANNABIX TECHNOLOGIES INC., Burnaby (CA); UNIVERSITY OF FLORIDA RESEARCH FOUNDATION, INC., Gainesville, FL (US)

(72) Inventors: Rajpaul ATTARIWALA, Vancouver (CA); Kulwant MALHI, Delta (CA); Richard YOST, Gainesville, FL (US); Jared BOOCK, Gainesville, FL (US); Michael T. COSTANZO, Gainesville, FL (US)

(21) Appl. No.: 16/082,220

(22) PCT Filed: Mar. 6, 2017
(86) PCT No.: PCT/CA2017/000042

§ 371 Date: Sep. 4, 2018

Related U.S. Application Data
(60) Provisional application No. 62/303,770, filed on Mar. 4, 2016.
(60) Provisional application No. 62/345,436, filed on Jun. 3, 2016.

(51) Int. Cl. A61B 5/00; G01N 27/62; G05D 16/06; G01N 33/497; G01N 1/40; A61B 5/08; A61B 5/097; A61B 5/087; A61B 5/091; A61B 10/00; B01D 46/00

(52) U.S. Cl. CPC A61B 5/4845; A61B 2010/0087; G05D 16/06; G01N 33/497; G01N 1/4077; A61B 5/082; A61B 5/097; A61B 5/087; A61B 5/091; A61B 10/00; B01D 46/00; G01N 2033/4975; G01N 2001/4088; A61B 2010/0009; G01N 27/624

(57) ABSTRACT

A system for collecting cannabis and the psychoactive component tetrahydrocannabinol or other controlled substances from a sample of exhaled breath. Single or multiple exhaled breaths are conditioned by removing contaminants, and regulating flow rate and/or pressure to collect a sample of tetrahydrocannabinol for timely local or remote analysis using FAIMS. The cannabis detection system comprises a containment trap for removing interfering materials from the breath of the subject and a collection component for sampling components of breath introduced into the system through the containment trap for FAIMS analysis to determine the level of THC or other controlled substance in the breath.

### TECHNICAL FIELD OF THE INVENTION

The present invention relates to detection devices for cannabis and other controlled substances. In particular, the present invention is directed to a portable device for the detection using High-Field Asymmetric Ion Mobility Spectrometry (“FAIMS”) of cannabis and other controlled substances from the exhaled breath of a subject.

### RELATED ART

Detection of cannabis and other controlled substances is commonly performed by urine, blood, or oral specimen sampling. These methods are frequently invasive and require complicated devices for analysis. Alcohol is a simple molecule which can be examined directly by an exhaled breath exam, most commonly by exhaling into an ion spectroscopy chamber. This method has proven reliable and is accepted by legal systems as a noninvasive method to quantify alcohol levels.

In this specification, “controlled substances” means any legal or illicit consumable drug, chemical, or other substance which is controlled by government regulation, and includes cannabis and alcohol. The terms “controlled substance(s)” and “drug(s)” are used interchangeably throughout.

Detection of drugs by an exhaled breath method has been proposed; however, the prior art technique proposed is generic and non-selective for multiple different illicit drugs. The proposed devices have been described for detection of drugs within the exhaled breath using a fluid collection or filtration system. Most of these devices describe a tube into which the subject exhales, which indiscriminately collects molecules of interest in either a liquid or filtration device. This liquid or fluid trapped within a filter is then sent to a laboratory for analysis, which may take several days. These devices lack methods for measurement of exhaled breath volumetric flowrate and for regulating pressure. With exhaled breath, each individual has the capability to exhale to different pressures, and if, for example, a filter based system is utilized to measure cannabis, an unregulated high pressure provided by a subject can tear apart the filter. Also, as each subject has a different amount of exhaled breath, it is important to be able to quantify the flowrate, or total volumetric flow, that has been breathed into the device.

The prior art devices which are designed to measure exhaled breath describe a mouthpiece which comprises a tube into which the subject exhales. This tube, without a rebreather valve, requires that the subject inhale through their nose, or remove the device from their lips to inhale. Prior devices designed for exhaled breath do not describe a method of removal of fluid or solid contaminants, such as by way of a spit trap to collect or remove oral fluids, which prevents oral fluid contaminants from reaching a gas material detection chamber.

### SUMMARY OF THE INVENTION

The present invention comprises a system which is designed to measure a subject's breath, remove fluid and/or contaminants, and collect a sample of tetrahydrocannabinol (THC) or other controlled substance in an entrapment container for detection of cannabis or other controlled substance use.

The invention includes a breathing tube with a rebreather valve, and a fluid and solid contaminant removal device. A pressure or volumetric flow measurement and regulating device is placed along the path of the exhaled breath pathway before or after a fixed or removable drug sample collecting chamber. The drug collecting chamber may contain a port for detection, preparation or collection of a sample of cannabis or other controlled substance. In the present invention, the method of detection is FAIMS.

The described device is designed to permit detection of cannabis use relatively quickly, thus allowing it to be used, for example, by police officers in the field, etc.

There is provided a controlled substance detection system for detecting one or more controlled substances in the breath of a subject, comprising: a containment trap for removing interfering materials from the breath of the subject; and a collection component for sampling components of breath introduced into the system through the containment trap for analysis to determine the presence of one or more controlled substances in the breath, in which the components of the breath sampled by the collection component are analyzed using FAIMS. The detection system may include a non-rebreather valve that allows the subject to inhale and exhale while introducing breath into the system, a pressure regulator for regulating the pressure of the breath introduced into the system, a flow measurement device for measuring a volumetric amount of breath introduced into the system by the subject, a mouthpiece for facilitating introduction of breath into the system by the subject, and a sampling port for allowing access to the components sampled by the collection component. The collection component may include a filter for trapping components of the breath sampled by the collection component. The containment trap may be a flap valve covering holes to allow fluids to be removed from the breath introduced into the system. The pressure regulator may be a balloon that inflates to regulate the pressure of breath introduced into the system. There may also be an appearance change material that changes appearance when in contact with a controlled substance.

The invention also provides a method of detecting cannabis or other controlled substance used by analyzing breath of a subject, comprising the steps of: removing contaminant material from the breath; collecting a sample of at least one component of the breath of the subject after the contaminant material has been removed therefrom; and analyzing the sample for the presence of one or more controlled substances, in which the components of the breath sampled by the collection component are analyzed using MAIMS. The method may include one or more of the additional steps of allowing the subject to inhale and exhale while introducing breath into the system, regulating the pressure of the breath introduced into the system, or measuring a volumetric amount of breath introduced into the system by the subject.

The invention further provides a detection system for detecting one or more controlled substances in the breath of a subject, comprising: a mouthpiece for facilitating introduction of breath into the system by the subject; a non-rebreather valve that allows the subject to inhale and exhale while introducing breath into the system; a containment trap for removing interfering materials from the breath of the subject introduced into the system; a pressure regulator for regulating the pressure of the breath introduced into the system a flow measurement device for measuring a volumetric amount of breath introduced into the system by the subject; a collection component for sampling components of breath introduced into the system through the containment trap; and a sampling port for allowing access to the components sampled by the collection component to facilitate analysis of the components sampled by the collection component to determine the presence of one or more controlled substances in the breath, in which the components of the breath sampled by the collection component are analyzed using FAIMS.

### BRIEF DESCRIPTION OF THE DRAWINGS

A detailed description of the preferred embodiments is provided below by way of example only and with reference to the following drawings, in which:

FIG. 1 is a schematic illustration showing an embodiment of the system to collect a cannabis or other drug sample from a breathing subject;

FIG. 2 is a flowchart illustrating a method for collecting a sample of cannabis or other controlled substance;

FIG. 3 is a schematic illustration showing an embodiment of the system to collect a cannabis or other drug sample from a breathing subject with an incorporated FAIMS spectrometer;

FIG. 4 is a flowchart illustrating a method for collecting a sample of cannabis or other controlled substance and measurement of controlled substances using a FAIMS spectrometer;

FIG. 5 depicts the ion mobility behavior of TUC;

FIG. 6 depicts the calibration curve of TI-IC using a hemispherical FAIMS electrode;

FIG. 7 depicts hemispherical FAIMS cell performance for THC and cannabinoid methyl ester;

FIG. 8 shows the mass spectrometry charts for THC and cannabinoid methyl ester;

FIG. 9 depicts the calibration curve of cocaine using a planar FAIMS electrode;

FIG. 10 depicts cocaine detection using a hemispherical FAIMS cell; and

FIG. 11 shows the mass spectrometry chart for cocaine.

### REFERENCE NUMERALS IN THE DRAWINGS

20 device for detection of cannabis or other drugs
24 Subject
30 mouth piece
32 intake nonrebreather valve
34 contaminant trap
36 drug sample collection and housing component
38 pressure measurement and/or regulator
40 liquid injection and or sampling port
42 volumetric flow measuring device
44 FAIMS spectrometer
50 breathing device
52 contaminant removal
54 exhaled breath flow conditioner
60 drug collection device
62 volume flow and pressure measurement device
64 sample preparation or collection device
66 FAIMS spectrometer

In the drawings, one embodiment of the invention is illustrated by way of example. It is to be expressly understood that the description and drawings are only for the purpose of illustration and as an aid to understanding, and are not intended as a definition of the limits of the invention.

### DETAILED DESCRIPTION

While this invention is susceptible of embodiments in many different forms, there are shown in the drawings and will herein be described in detail, preferred embodiments of the invention with the understanding that the present disclosure is to be considered as an exemplification of the principles of the invention and is not intended to limit the broad aspects of the invention to the embodiments illustrated.

FAIMS is an ion mobility spectrometry technique which exploits differences in ion mobility at high electric field strength. FAIMS has been important in the detection of substances of interest to law enforcement and public safety, including narcotics. In the present invention, a partial ovoidal FAIMS electrode is used to permit detection of cannabis and other controlled substances. U.S. Pat. No. 8,237,118 entitled Partial Ovoidal FAIMS Electrode describes the FAIMS technology used in the present invention, and is incorporated herein in its entirety by reference.

Cannabis detection by breath requires many components to separate raw cannabis and its metabolized forms from the psychoactive component tetrahydrocannabinol (THC).

The present invention may be used to detect controlled substances in two modes. Firstly, the device of the present invention as shown in FIG. 2 may be used to collect and access a sample of one or more controlled substances from a subject's breath. The sample may then be analyzed separately using FAIMS technology.

According to the second mode, the FAIMS spectrometer may be incorporated into the portable breathalyzer, as is shown in the embodiment of FIG. 3.

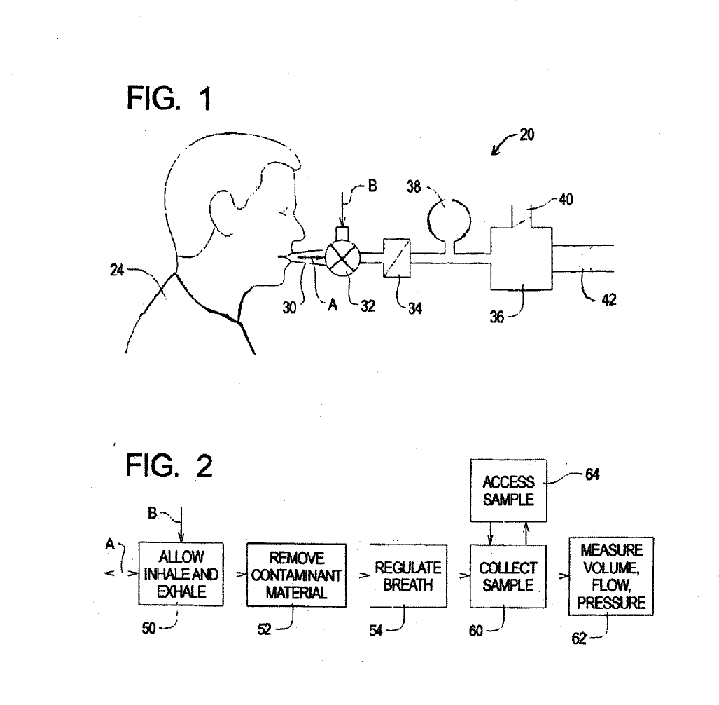

The embodiment of the invention described in FIG. 1 consists of a portable handheld system for detection of cannabis or other drugs 20, where the psychoactive component of cannabis, THC, or other controlled substance, is detected from the breath of a subject 24.

FIG. 2 is the block diagram illustrating a method for detecting and/or measuring THC or other drugs in the breath of the subject 24 using the embodiment of the device depicted in FIG. 1. The method depicted in the block diagram of FIG. 2 may be implemented by the portable detection system. 20 of FIG. 1 or by any device or system capable of performing at least some combination of the steps described in FIG. 1.

The example portable controlled substance detection system 20 comprises a mouthpiece 30, an intake non-rebreather valve 32, a contaminant trap 34, a collection and housing component for THC or other drugs 36, a pressure measurement and/or regulator component 38, a liquid injection/sampling port 40, and a volumetric flow measuring component 42. The detection system 20 is comprised of either disposable, or aseptic cleanable reusable components or as a single whole device.

The flowchart of FIG. 2 outlines the method for detection of THC or other drugs by outlining the direction materials pass through a device for drug detection such as the portable detection system 20 as generally described above. The detection method depicted in FIG. 2 comprises the steps of providing a breathing device at block 50, removing contaminant material at block 52, conditioning the exhaled breath flow at block 54, collecting the THC or other drugs at the block 60, measuring at least one and typically all of volume, flow, and pressure at block 62, and collecting or preparing a sample at block 64.

The components and materials used by the steps in the flowchart of FIG. 2 are designed to provide to a drug collection device such as the drug collection component 36 of the example portable detection system 20 described above, at block 60, an appropriate sample of exhaled breath from which THC or other drugs content may be detected and/or measured.

Referring now more specifically to FIG. 1 of the drawing, it can be seen that the example detection system 20 is coupled to the subject 24 via the mouthpiece 30. This mouthpiece 30 is directly connected to a non-rebreather valve 32 which allows the subject 24 to both inhale and exhale through the mouthpiece 30 without the subject 24 removing lips from the mouthpiece 30 to inhale and without requiring the subject 24 to breathe through the nose to inhale. The air exchange step performed by the example mouthpiece 30 and non-rebreather valve 32 is described at the block 50 in the flowchart of FIG. 2. Arrows A and B in FIGS. 1 and 2 show air entering and exiting the system, respectively, thereby allowing the subject 24 to inhale and exhale through the mouthpiece 30, with the exhaled breath being redirected into the contaminant trap 34.

The example contaminant trap 34 consists of a device that allows interfering materials, such as vapor, fluid, and/or solids, to be removed from the exhaled breath from the subject 24 and allows exhaled THC or other drugs-containing breath to pass through unrestricted. The example trap 34 thus removes contaminants from the exhaled breath as performed by the removing contaminant material step shown at block 52 in FIG. 2. The containment trap 34 can be removed to access liquid contaminants which may include metabolized or unmetabolized components of cannabis or other drugs to remove components of exhaled breath that would interfere with the detection of THC or other drugs in the collection device 36. The example containment trap 34 may be a contaminant removal component comprising a flap valve covering holes that allows oral fluid to pass out of the device or to be collected in another chamber.

The degree of effort of exhalation by subjects such as the subject 24 is variable, which results in the exhaled breath occurring at various pressures. To provide the THC or other drugs collection component 36 with a suitable pressure or flow rate, a pressure regulator and or measuring device 38 is placed in line with the exhaled breath. The example THC or other drugs collection component 36 prepares the exhaled breath to be appropriately collected as shown at the step corresponding to block 54 in the flowchart of FIG. 2. The pressure measuring and/or regulating component 38 can be placed anywhere beyond the mouthpiece to control pressure input into or from the device. A wide variety of pressure regulating or measuring devices can be utilized, and an example may consist of a balloon which expands with high pressure exhaled breath, and collapses to deliver exhaled breath to the detection component of the device at a controlled pressure.

The drug collection component 36 may consist of a vacuum, gas and/or liquid filled chamber with a filter or sensor that is capable of collecting or detecting THC or other drugs. The example drug collection device 36 may be directly accessed by a sampling port 40 for either sampling or filling with gas or liquid materials as shown by block 64 in FIG. 2. The drug collection component 36 in whole or in part may be removed from the detection system 20. According to one embodiment, the drug collection component 36 may be a filter or cartridge that can be removed for sampling via FAIMS. The port 40 may be used to prepare the filtered sample for either of these methods.

FAIMS provides a highly selective means of separating ions at atmospheric pressure. Incorporating FAIMS technology into a handheld, portable device, provides an elegant solution for detection of THC and other controlled substances in the exhaled breath of a subject.

FAIMS separates ions based on their unique change in mobility under high and low electric fields. Reference mobilities may be established for THC, cocaine, alcohol, and other controlled substances. The device of the present invention may be used to separate and identify the concentration of the controlled substance components in a subject's breath using FAIMS.

In the embodiment of the device of the invention shown in FIG. 3, a FAIMS spectrometer 44 is incorporated into the device to allow detection of controlled substances immediately upon sample collection.

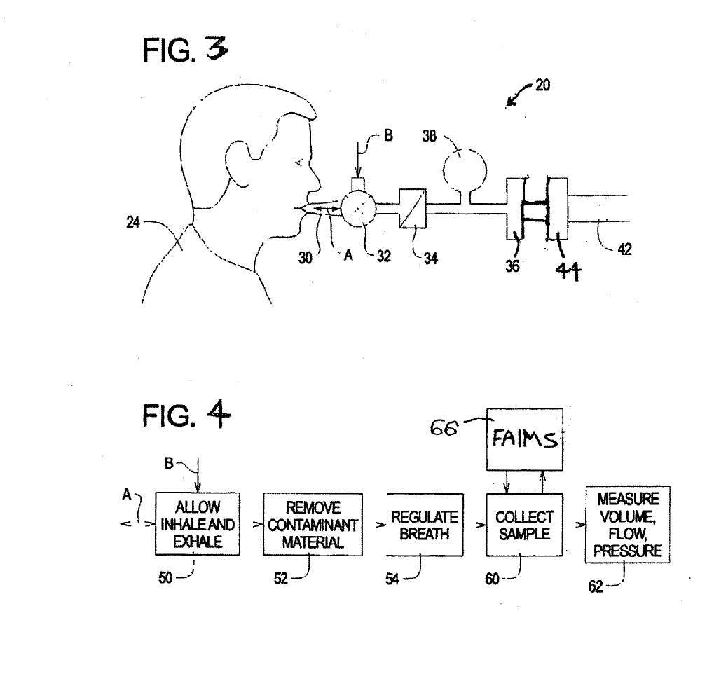

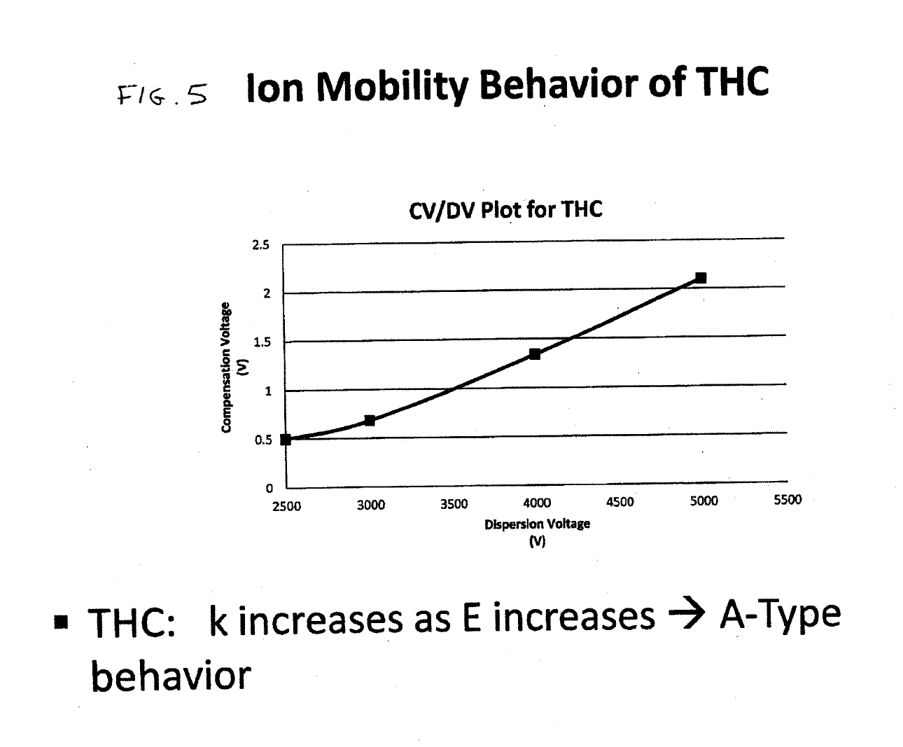

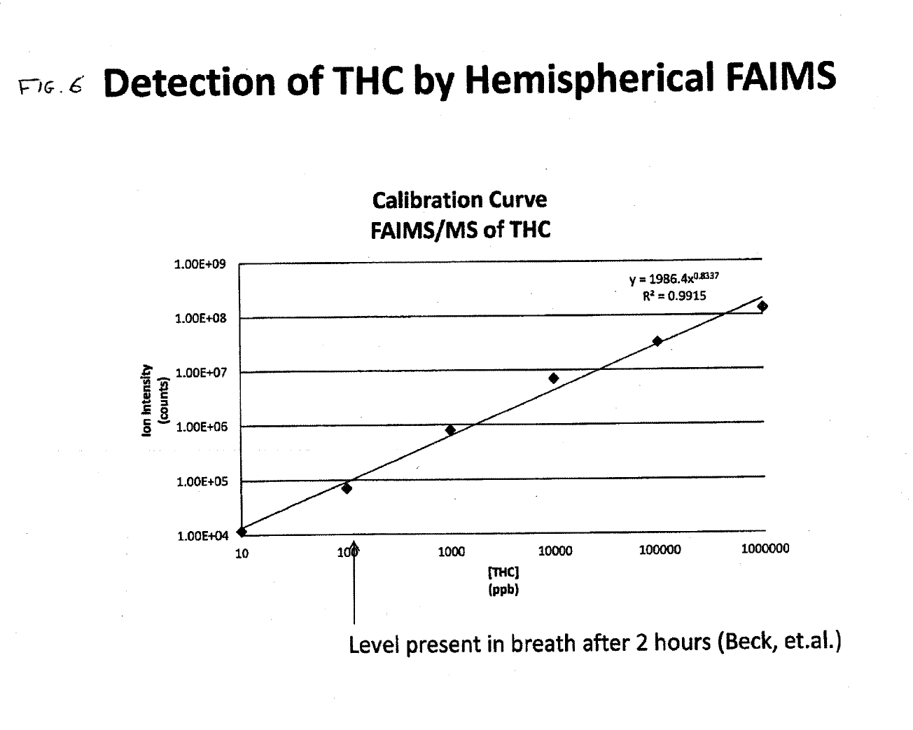

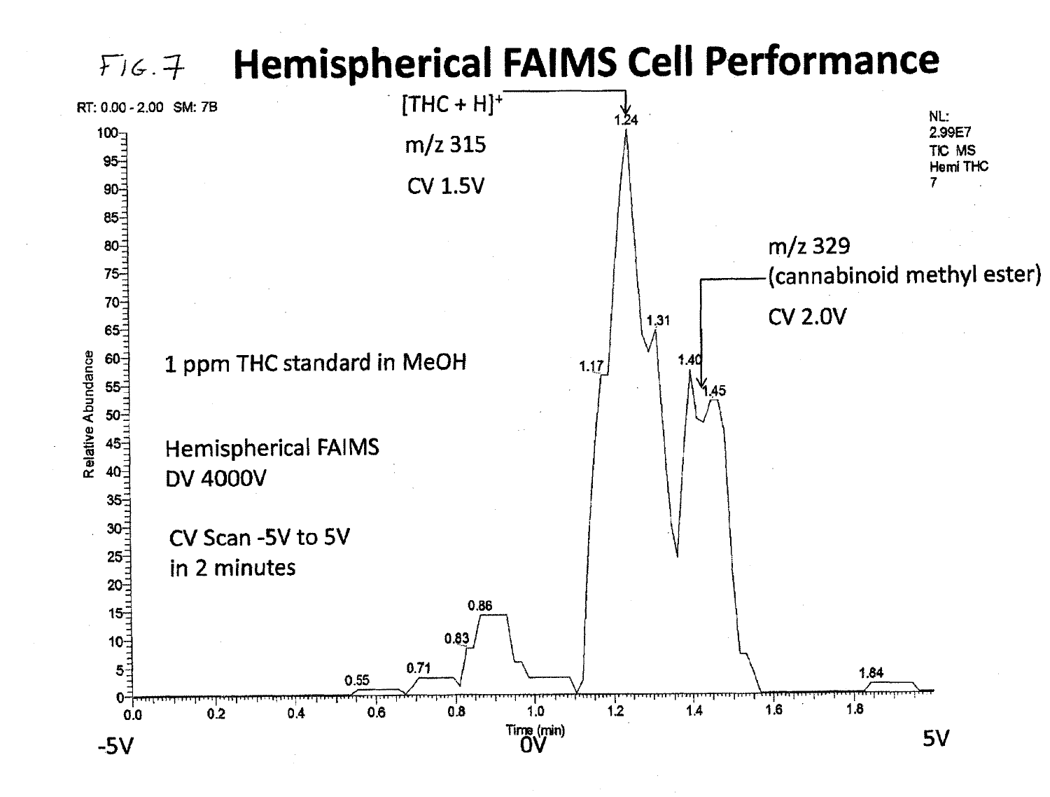

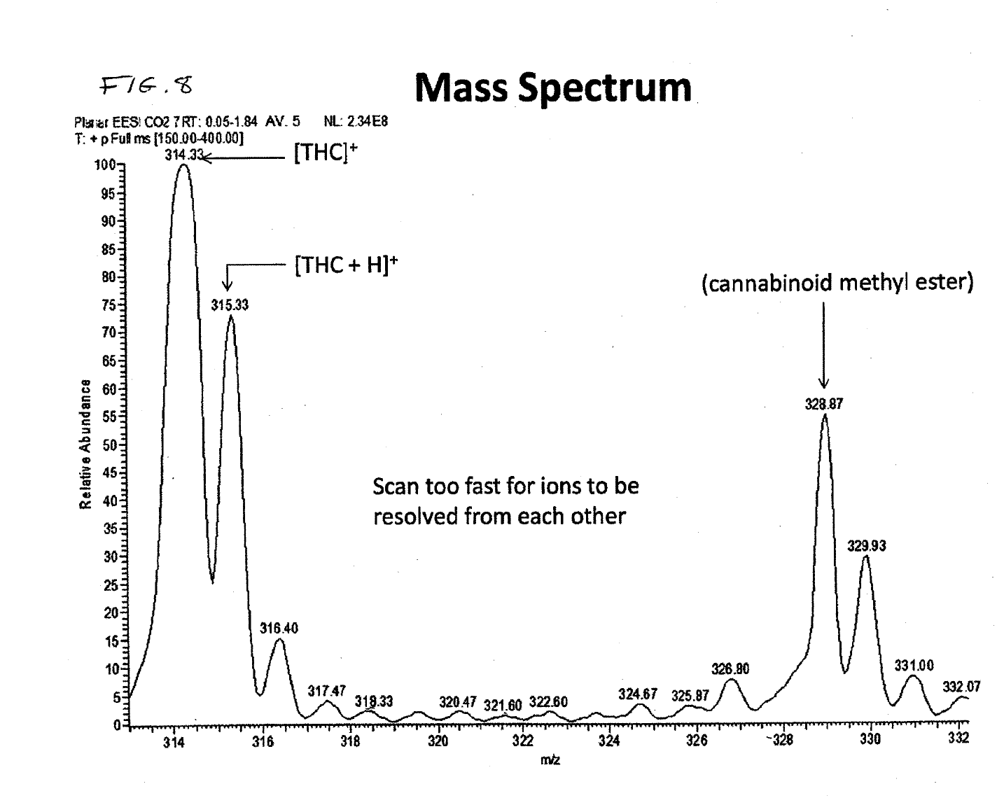

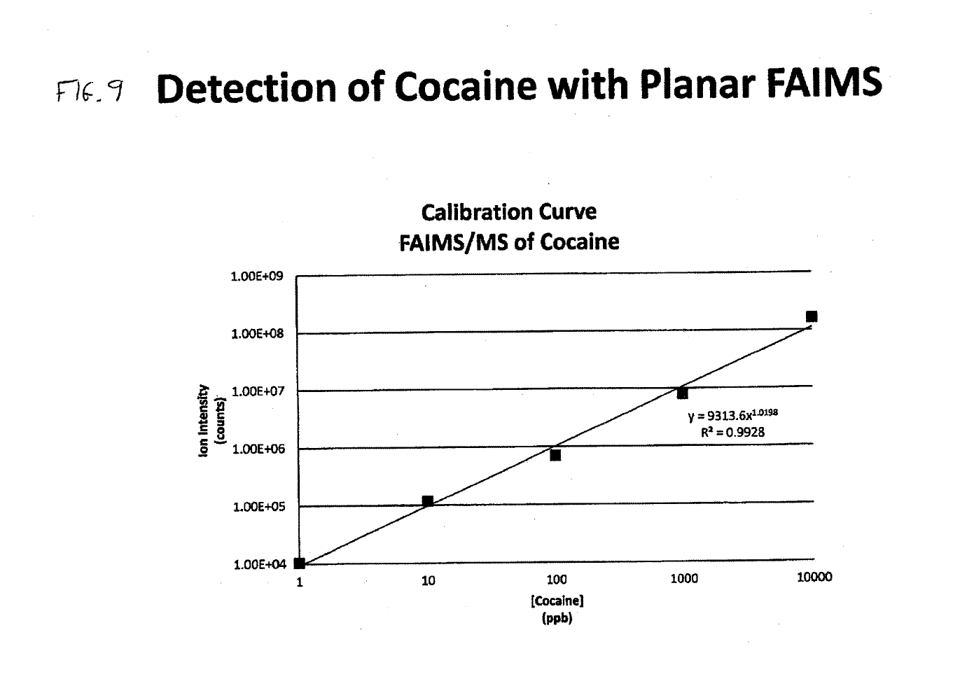

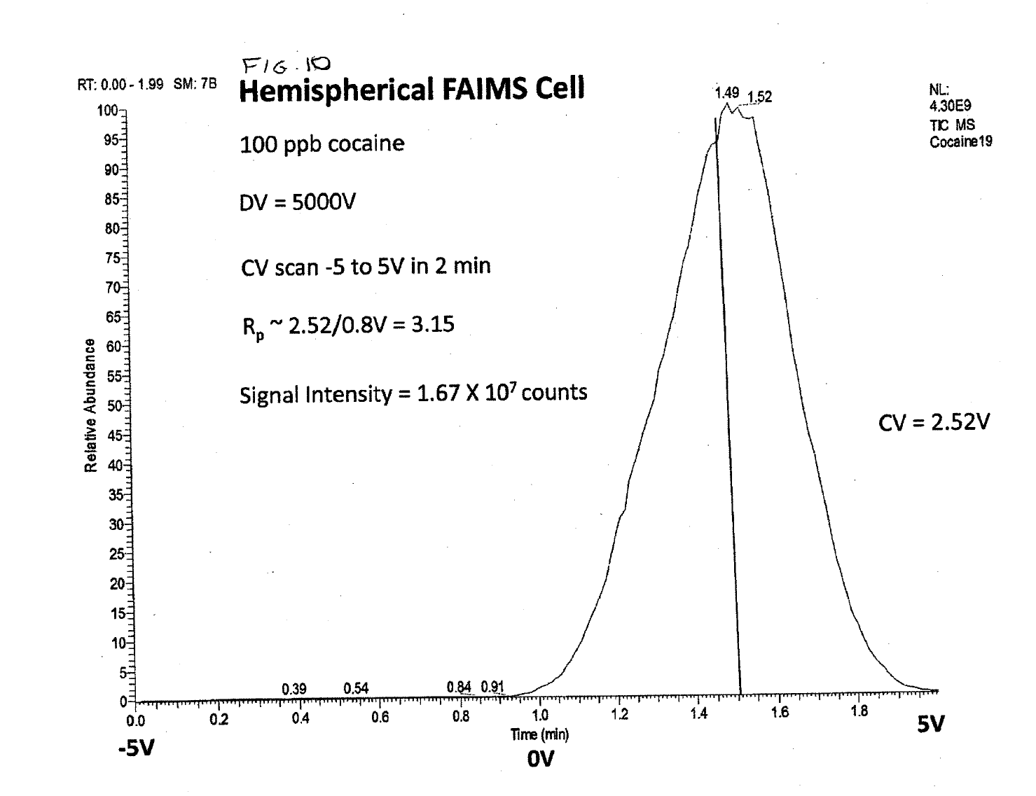

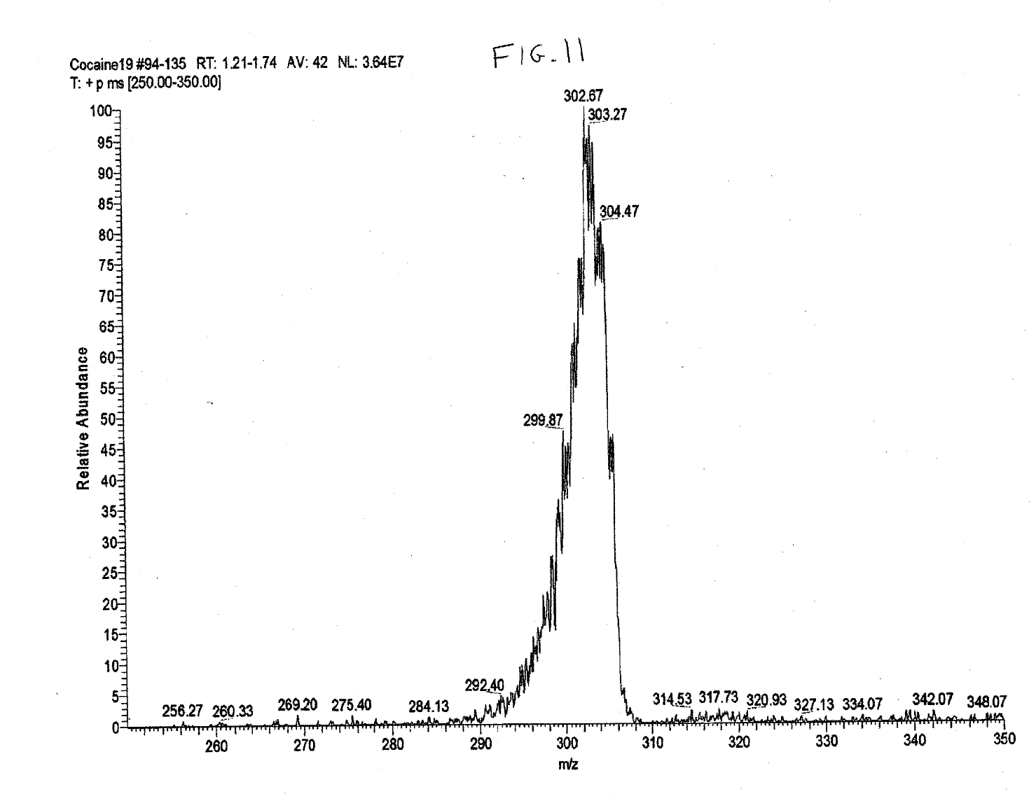

FIG. 4 shows the method of collection and measurement of controlled substances using the embodiment of the device of the invention shown in FIG. 3. Following sample collection, the sample is introduced into a FAIMS spectrometer 66 for measurement of the controlled substances of interest.

In line with the exhaled breath flowchart as outlined in FIGS. 1 and 3, a volumetric flow measuring device 42 is placed to quantify the amount of exhaled breath provided by the subject 24. The location of this measurement device 62 as shown in the flowchart of FIGS. 2 and 4 can be placed anywhere along the pathway of the exhaled breath, or may be coupled with the step of conditioning the exhaled breath flow shown by block 54 and/or the pressure measurement or regulator device 38. The purpose for recording the volume of breath passing through the device is to enable a way to measure how much exhaled breath has been input into the system 20. Each individual, based on their lung capacity and/or effort, can exhale different volumes.

A volumetric flow rate measurement device incorporated into the system for cannabis detection provides a means to quantify this amount per breath, and/or as a total sum during use of the device. This ensures that sufficient exhaled breath is delivered to the drug collection component 36 to allow detection of cannabis, THC or other drugs. A variety of different flow meters can be used as the volumetric flow measuring device 42. One example is a rotating blade which spins with exhaled breath and records the volume of flow that is exhaled. A second example would be a diaphragm that detects air pressure and converts air pressure to flow volume.

In one embodiment, the device can provide results of THC or other drugs detection in seconds or minutes based on how the drug detection device 20 and/or drug collection analysis method are implemented.

In another embodiment, the device can be implemented or equipped to correlate a measured or detected THC or other drug sample to a specific subject. For example, a DNA fingerprint method can be added to the contaminant trap 34, or elsewhere inline, to detect and/or monitor who is utilizing the device (e.g., through use of the subject's saliva or other DNA sample). In this case, the THC or other drugs sample measurement and the DNA sample are stored together and/or cross-reference in a way that ensures that a particular THC or other drug sample measurement and DNA sample are positively associated with each other for evidentiary purposes if necessary.

The use of FAIMS technology is suitable for detection of cannabis and other controlled substances and their metabolites in human breath. FAIMS has been able to detect levels of THC equivalent to the level remaining in human breath 2 hours after inhalation. Low parts-per-billion nicotine has also been successfully detected.

Several embodiments of the invention have been described. It should be understood that the concepts described in connection with one embodiment of the invention may be combined with the concepts described in connection with another embodiment (or other embodiments) of the invention.

While an effort has been made to describe some alternatives to the preferred embodiment, other alternatives will readily come to mind to those skilled in the art. Therefore, it should be understood that the invention may be embodied in other specific forms without departing from the spirit or central characteristics thereof. The present examples and embodiments, therefore, are to be considered in all respects as illustrative and not restrictive, and the invention is not intended to be limited to the details given herein.

1. A controlled substance detection system for detecting one or more controlled substances in the breath of a subject, comprising: a containment trap for removing interfering materials from the breath of the subject; and a collection component for sampling components of breath introduced into the system through the containment trap for analysis to determine the presence of one or more controlled substances in the breath, in which the components of the breath sampled by the collection component are analyzed using FAIMS.

2. A detection system as recited in claim 1, further comprising a non-rebreather valve that allows the subject to inhale and exhale while introducing breath into the system.

3. A detection system as recited in claim 1, further comprising a pressure regulator for regulating the pressure of the breath introduced into the system.

4. A detection system as recited in claim 1, further comprising a flow measurement device for measuring a volumetric amount of breath introduced into the system by the subject.

5. A detection system as recited in claim 1, further comprising a mouthpiece for facilitating introduction of breath into the system by the subject.

6. A detection system as recited in claim 1, further comprising a sampling port for allowing access to the components sampled by the collection component.

7. A detection system as recited in claim 1, in which the collection component further comprises a filter for trapping components of the breath sampled by the collection component.

8. A detection system as recited in claim 1, in which the containment trap comprises a flap valve covering holes to allow fluids to be removed from the breath introduced into the system.

9. A detection system as recited in claim 3, in which the pressure regulator comprises a balloon that inflates to regulate the pressure of breath introduced into the system.

10. A detection system as recited in claim 1, further comprising an appearance change material that changes appearance when in contact with a controlled substance.

11. A method of detecting cannabis or other controlled substance used by analyzing breath of a subject, comprising the steps of: removing contaminant material from the breath; collecting a sample of at least one component of the breath of the subject after the contaminant material has been removed therefrom; and analyzing the sample for the presence of one or more controlled substances, in which the components of the breath sampled by the collection component are analyzed using FAIMS.

12. A method as recited in claim 11, further comprising the step of allowing the subject to inhale and exhale while introducing breath into the system.

13. A method as recited in claim 11, further comprising the step of regulating the pressure of the breath introduced into the system.

14. A method as recited in claim 11, further comprising the step of measuring a volumetric amount of breath introduced into the system by the subject.

15. A detection system for detecting one or more controlled substances in the breath of a subject, comprising: a mouthpiece for facilitating introduction of breath into the system by the subject; a non-rebreather valve that allows the subject to inhale and exhale while introducing breath into the system; a containment trap for removing interfering materials from the breath of the subject introduced into the system; a pressure regulator for regulating the pressure of the breath introduced into the system a flow measurement device for measuring a volumetric amount of breath introduced into the system by the subject; a collection component for sampling components of breath introduced into the system through the containment trap; and a sampling port for allowing access to the components sampled by the collection component to facilitate analysis of the components sampled by the collection component to determine the presence of one or more controlled substances in the breath, in which the components of the breath sampled by the collection component are analyzed using FAIMS.

16. A detection system as recited in claim 15, in which the collection component further comprises a filter for trapping components of the breath sampled by the collection component.

17. A detection system as recited in claim 15, in which the containment trap comprises a flap valve covering holes to allow fluids to be removed from the breath introduced into the system.

18. A detection system as recited in claim 15, in which the pressure regulator comprises a balloon that inflates to regulate the pressure of breath introduced into the system.

19. A detection system as recited in claim 15, further comprising an appearance change material that changes appearance when in contact with a controlled substance.
